(19) United States
(12) Patent Application Publication
(10) Pub. No.: US 2016/0184565 A1
(43) Pub. Date: Jun. 30, 2016

(54) DELIVERY OF MEDICANTS UNDER CONTINUOUS NEGATIVE PRESSURE DRESSING

(71) Applicant: PLUROGEN THERAPEUTICS, LLC, Norristown, PA (US)

(72) Inventor: George RODEHEAVER, Charlottesville, VA (US)

(21) Appl. No.: 14/910,533

(22) PCT Filed: Aug. 7, 2014
(86) PCT No.: PCT/US14/50212

§ 371 Date: Feb. 5, 2016

Related U.S. Application Data
(60) Provisional application No. 61/863,160, filed on Aug. 7, 2013.

(51) Int. Cl. A61M 35/00; A61M 1/00

(52) U.S. Cl. CPC A61M 35/00; A61M 1/0023

(57) ABSTRACT

Disclosed herein are devices for delivery of an antimicrobial to a patient. Such devices comprise an antimicrobial delivery device comprised of a rigid reservoir having a proximal surface, an interstitial space, and a distal surface; and a manifold positioned underneath the rigid reservoir. This antimicrobial delivery device may also include an injection port. Systems and methods for delivering a medicant to a wound under continuous negative pressure are also disclosed.

### CROSS-REFERENCE TO RELATED APPLICATIONS

Not applicable

### GOVERNMENT INTERESTS

Not applicable

### PARTIES TO A JOINT RESEARCH AGREEMENT

Not applicable

### INCORPORATION BY REFERENCE OF MATERIAL SUBMITTED ON A COMPACT DISC

Not applicable

### BACKGROUND

Wound management encompasses chronic and acute situations where assistance is needed for the natural healing process. Maintaining an environment that allows a wound to heal optimally is essential. Therefore, systems and methods to prevent and mitigate infection, sloughing, necrosis, and chronic seroma cavities are required.

Surgical wound drainage is a key element in facilitating these healing processes. Mechanical systems include suction devices. However, such devices are prone to clog or alternatively drain too quickly. It is necessary to prevent infection during the drainage process without affecting the drain system.

Thus, there is a need for improved delivery of medicants for the prevention or treatment of infections during fluid removal of a wound. There also remains a need for a device and method that is cost-effective, patient-friendly, clinician-friendly, and compatible with existing drain systems.

### SUMMARY OF THE INVENTION

Disclosed herein are devices for delivery of an antimicrobial to a patient as well as systems and methods for delivering a medicant to a wound under continuous negative pressure. In some embodiments, an antimicrobial delivery device comprises a rigid reservoir and a manifold positioned underneath the surface of the rigid reservoir proximal to a wound. In other embodiments, an antimicrobial delivery device comprises a rigid reservoir, a manifold positioned underneath the surface of the rigid reservoir proximal to a wound, and an injection port positioned on top of the manifold and rigid reservoir. In some embodiments, a method of delivering a medicant to a wound under continuous negative pressure is disclosed. In some embodiments, a configurable medicant delivery system comprising an antimicrobial delivery device, a wound, a means of generating negative pressure, and delivering the medicant under negative pressure to a wound is disclosed.

### BRIEF DESCRIPTION OF THE FIGURES

FIG. 1 illustrates a cross-sectional view of a manifold.

FIG. 2A illustrates a cross-sectional view of an embodiment of an antimicrobial delivery device.

FIG. 2B illustrates a cross-sectional view of an additional embodiment of an antimicrobial delivery device having an injection port.

### DETAILED DESCRIPTION

Disclosed herein are devices for delivery of an antimicrobial to a patient and systems and methods for delivering a medicant to a wound under continuous negative pressure. Before the present devices and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

It must be noted that as used herein, and in the appended claims, the singular forms “a,” “an,” and “the” include plural reference unless the context clearly dictates otherwise. Thus, for example, reference to a “medicant” comprises a medicament, medication, medicine, pharmaceutical, drug, and the like used for healing, treating, altering, improving, restoring, relieving, and/or curing a particular condition, disease, or mental or physical state, which includes the active ingredient or combination of active ingredients and inactive ingredients infused into an expedient or dissolved in some other carrier. Unless defined otherwise, all technical and scientific terms used herein have the same meanings as commonly understood by one of ordinary skill in the art. As used herein, the term “comprising” means “including, but not limited to.”

As used herein, all claimed numeric terms are to be read as being preceded by the term, “about,” which means plus or minus 10% of the numerical value of the number with which it is being used. Therefore, a claim to “50%” means “about 50%” and encompasses the range of 45%-55%.

The terms “treating” or “treatment” include the administration of the disclosed compositions thereby alleviating the symptoms, or eliminating the disease, condition, or disorder as well as acting as prophylaxis against injury and wound sequellae.

The term “biocompatible” as used herein refers to a composition being compatible with living tissue such as in an implant. While biocompatibility can be measured via any number of parameters, compositions that do not elicit an immune response (or only a minimal response) are consistent with being biocompatible. Similarly, compositions that are not toxic to an organism are also consistent with being biocompatible.

The term “negative pressure” therapy includes any treatment where any form of negative pressure is applied to a wound or injury to ensure negative pressure and, thereby, adequate drainage.

The term “reservoir” includes any substrate, structure, or container in which high proportions of a fluid or gel are immobilized reliably and stably. For example, a reservoir can comprise a medicant for delivery to a wound or injury site. The term “manifold” is a device that creates fluid communication between a reservoir and a delivery site.

The term “tissue” refers to an aggregate of morphologically similar cells with associated intercellular matter that may act together to perform one or more specific functions in the body of an organism including a human. The term “tissue” also encompasses organs comprising one or more tissue types.

The term “wound” refers to any damage to a tissue in a living organism, including human organisms. The tissue may be an internal tissue such as an internal organ or an external tissue such as the skin. The wound may be of one tissue or multiple tissues adjacent to one another. The term “chronic wound” generally refers to a wound that has not healed within 30 days. The term “promoting wound healing” generally refers to enabling reconstitution of the normal physiologic barrier function of a tissue.

The term “absorptive material” refers to any sponge, foam, hydrogel, or any other material that can absorb or retain a liquid or gel. The term “sponge” refers to an elastic porous mass that can absorb or retain a liquid or gel. The term “foam” refers to a substance which has gas-solid structures, such as a multitude of gas cells inside a solid matrix. As used herein, the term “hydrogel” means a two- or multicomponent system including a three-dimensional network of polymer chains and water that may fill the spaces between the macromolecules.

The term “anti-microbial” refers to any bactericidal, bacteriolytic, or bacteriostatic agent.

The term “pore” refers to any opening in an area that allows for any liquid, gel, or small particles to pass through the area. Pore can be introduced into any material, device, or surface.

The term “proximal” refers to a location that is next to or nearest the point of interest. For example, the spatial orientation of a medical device in relation to a wound, proximal refers to the location of the device nearest the wound. The term “distal” refers to a location that is furthest from the point of interest. For example, the spatial orientation of a medical device in relation to a wound, distal refers to the location of the device farthest from the wound. The term “interstitial space” refers to a space or gap between two layers of an object or tissue.

Reference in the specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment.

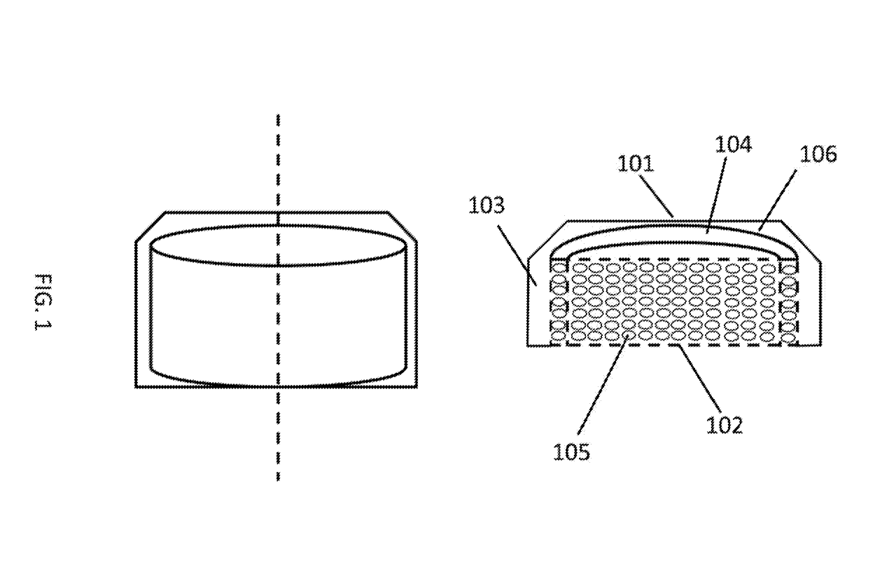

FIG. 1 illustrates an embodiment of a reservoir that has a distal surface 101, a proximal surface 102, and interstitial space 103. The reservoir may also contain an impermeable layer 104 on the distal surface in order to prevent diffusion of any medicant, such as an antimicrobial agent or a growth factor, out of the distal surface 101. Additionally, the reservoir may have a manifold 106 that is positioned within the interstitial space 103 of the reservoir. The manifold 106 may contain efflux pores 105 to allow for flow of any medicant out of the proximal surface 102 of the reservoir. The efflux pores 105 may have a diameter of about 500 nanometers, about 1 micrometer, about 10 micrometers, about 50 micrometers, about 100 micrometers, about 200 micrometers, about 300 micrometers, about 400 micrometers, about 500 micrometers, about 600 micrometers, about 700 micrometers, about 800 micrometers, about 900 micrometers, about 1000 micrometers and any ranges between any of these values (including endpoints).

Where efflux pores 105 are semi-permeable, flow can be by means of diffusion. The efflux pores 105 may have a molecular weight cutoff (MWCO) wherein the MWCO is defined as the molecular weight at which about 80% of the medicant is prevented from diffusing out of the manifold across the proximal surface. The efflux pores 105 may have a MWCO of about 1,000 kilodaltons (kD), about 2,000 kD, about 5,000 kD, about 10,000 kD, about 20,000 kD, about 30,000 kD, about 40,000 kD, about 50,000 kD, about 100,000 kD, about 200,000 kD, about 300,000 kD, about 400,000 kD, about 500,000 kD, about 600,000 kD, about 700,000 kD, about 800,000 kD, about 900,000 kD, about 1,000,000 kD, and any range between any of these values (including endpoints).

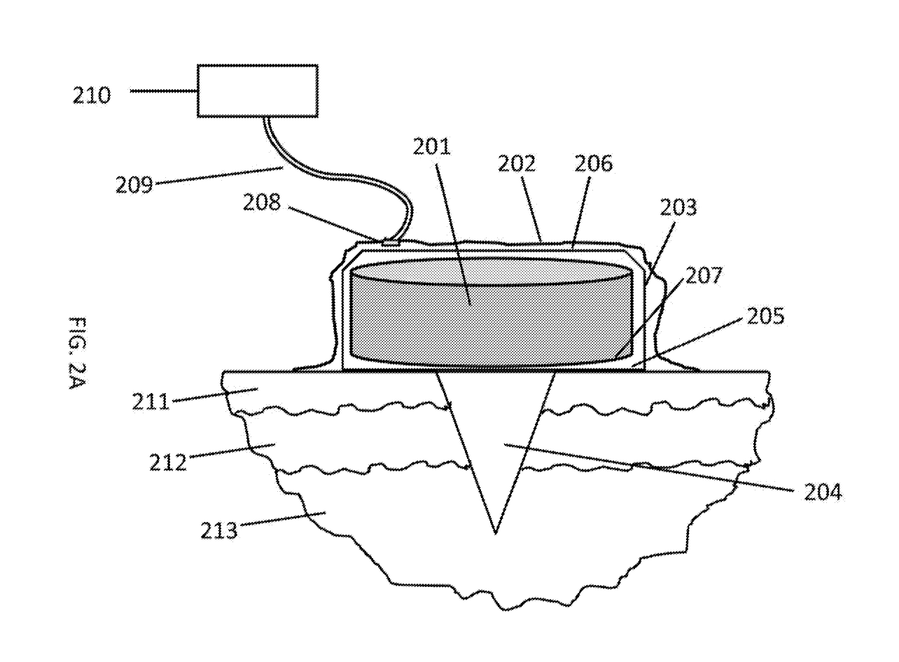

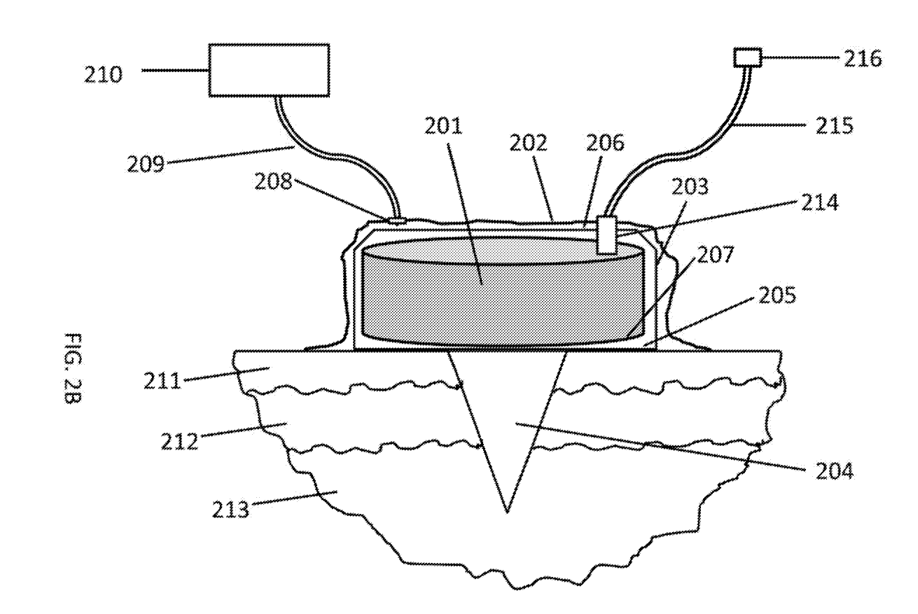

In some embodiments, the antimicrobial delivery device may further include a negative pressure conduit positioned to create suction at the distal surface of a wound drape. FIG. 2A represents an embodiment of an antimicrobial delivery device, that includes a wound drape 202 to encase the device and a negative pressure conduit 208, positioned over a wound 204. The wound drape 202 may act as a securing material to position the device over a desired location on a patient. The desired location may be a wound 204 of varying size. The desired location may be a plurality of wounds of varying size. The wound drape 202 may be secured using any form of adhesive substance. The wound drape 202 may encase the distal surface 206 of the rigid reservoir 203 and the side of the rigid reservoir 203. The rigid reservoir 203, manifold 201, and wound drape 202, may be of any range of sizes to accommodate a variety of wound diameters and depths or a plurality of wounds. In some embodiments, the wound 204 represents a wound 204 that extends from the epidermis 211, dermis 212, and into the subcutaneous tissue 213. In some embodiments, the wound 204 extends from the epidermis 211 into the dermis 212. In other embodiments, the wound 204 only extends into the epidermis 211.

In some embodiments, the negative pressure conduit 208 may be attached to a first end of a tube 209 and a source of negative pressure 210 may be attached to a second end of the tube 209. The negative pressure conduit 208 may be positioned at the distal surface 206 of the wound drape 202. The proximal surface 205 of a reservoir may have a semipermeable membrane 207 which is positioned to come into contact with the wound 204. The negative pressure conduit 208 may permit drainage of the wound 204 during the healing process. The source of negative pressure 210 may provide continuous negative pressure, thus resulting in continuous wound drainage. In some embodiments, during the delivery of continuous negative pressure, the medicant flows from the manifold 201 into the wound to aid in the healing process of the wound 204. In other embodiments, the source of negative pressure 210 may provide intermittent negative pressure, thus resulting in intermittent wound drainage. In some embodiments, during the intermittent negative pressure, the medicant flows from the manifold 201 into the wound to aid in the healing process of the wound 204 in between the intermittent negative pressure.

The manifold 201 may be any material that has structural elements which form channels to allow for flow of any liquid, gel, foam, or combinations thereof. In some embodiments, the manifold 201 may be a foam, sponge, hydrogel, semi-permeable membrane cell, any similar composition, or combinations thereof. In other embodiments, the manifold 201 may also be a combination or layering of materials, such as, but not limited to, a dense foam layer on the top, a less dense foam layer adjacent to the dense foam layer, and a semi-permeable membrane cell adjacent to the less dense foam layer to create a gradient of flow channels where any liquid, gel, or foam will diffuse at different rates in each layer. The manifold 201 may control the flow rate of medicant into the wound by varying combinations or layering of materials.

The manifold 201 may be a foam comprised of efflux pores 105 as depicted in FIG. 1. The foam may have a compressive strength of about 0.01 megapascal (MPa), about 0.02 MPa, about 0.05 MPa, about 0.1 Mpa, about 0.2 MPa, about 0.3 MPa, about 0.4 MPa, about 0.5 MPa, about 0.75 MPa, about 1.0 Mpa, about 2.0 MPa, about 3.0 MPa, about 5.0 MPa, about 10 MPa, about 15 MPa, about 20 MPa, about 25 MPa, about 30 MPa, about 35 MPa, about 40 MPa, about 50 MPa, about 60 MPa, about 70 MPa, about 80 MPa, about 90 MPa, about 100 MPa, about 110 MPa, about 120 MPa, and any range between any of these values (including endpoints).

The manifold 201 may be a sponge comprised of efflux pores 105 as depicted in FIG. 1. The manifold 201 may be a hydrogel comprised of efflux pores 105. The hydrogel may include, but not limited to, polyvinyl alcohol (PVA), polyhydroxyethyl methacrylate, polyvinyl pyrrolidone, polyacrylamide, polyacrylic acid, hydrolyzed polyacrylonitrile, polyethyleneimine, ethoxylated polyethyleneimine, polyallylamine, polyglycols, and the like and combinations thereof. The manifold 201 may be a semi-permeable membrane cell comprised of efflux pores 105. In some embodiments, the efflux pores 105 may have a pre-defined pore size. In other embodiments, the efflux pores 105 may have a pre-defined MWCO.

According to a second embodiment, as seen in FIG. 2B, the antimicrobial delivery device may have an injection port 214 positioned on the distal surface 206 of the rigid reservoir 203. The injection port 214 may also be connected at one end to an injection tube 215. In some embodiments, the injection tube 215 may have a syringe attachment 216 at a second end of the injection tube 215. The injection port 214 and injection tube 215 may be used to administer a medicant to the manifold 201. The injection port 214 and injection tube 215 may be used to administer a medicant to the manifold 201 while the antimicrobial delivery device is under negative pressure. The injection port 214 and injection tube 215 may be used to administer a medicant to the manifold 201 while the antimicrobial delivery device is under continuous negative pressure.

A system for a delivering a medicant is disclosed herein. For example, a configurable medicant delivery system may have an antimicrobial delivery device, a wound 204, a means of generating negative pressure between the antimicrobial delivery device and wound 204 thereby creating suction at the wound 204; and delivering the medicant under negative pressure to the wound 204. In some embodiments, the antimicrobial delivery device may have a rigid reservoir 203 and a manifold 201. In other embodiments, the antimicrobial delivery device may have a manifold 201 without a separate rigid reservoir 203. In yet other embodiments, the antimicrobial delivery device may have a rigid reservoir 203 without a manifold 201. In some embodiments, the antimicrobial delivery device is placed over the wound 204.

In some embodiments, the manifold 201 in the medicant delivery system may be a foam, a sponge, a hydrogel, a semi-permeable membrane cell, and combinations thereof. In other embodiments, the manifold 201 may be a compressible foam. In some embodiments, the manifold 201 may be of the same design and composition as seen in FIG. 1.

A method of delivering a medicant to a wound under continuous negative pressure is disclosed herein. For example, a method of delivering a medicant to a wound 204 under continuous negative pressure may comprise sealing a rigid reservoir comprising a manifold 201 over a suctioned wound surface and effluxing the medicant while providing continuous negative pressure to the wound from a source of negative pressure 210. In some embodiments, the manifold 201 may be proximal to the wound surface. In some embodiments, a manifold 201 may be positioned underneath the proximal surface 205 of a rigid reservoir 203. The rigid reservoir 203 may have the proximal surface 102, the interstitial space 103, and the distal surface 101 as depicted in FIG. 1. In other embodiments, a manifold 201 may be positioned within the interstitial space 103 of a rigid reservoir 203. In further embodiments, the method of delivery may comprise an antimicrobial delivery device having a manifold 201 and a rigid reservoir 203. In other embodiments, the method of delivery may comprise an antimicrobial delivery device having a manifold 201. In yet further embodiments, the method of delivery may comprise an antimicrobial delivery device additionally having an injection port 214 positioned on the distal surface 206 of a manifold 201 and a rigid reservoir 203.

Various embodiments are directed to an antimicrobial delivery device comprising a rigid reservoir 203 having the proximal surface 102, the interstitial space 103, and the distal surface 101 as depicted in FIG. 1, additionally including a manifold 201 positioned underneath the proximal surface of the rigid reservoir 203. The manifold 201 may also be positioned within the interstitial space 103 of the rigid reservoir 203. The manifold 201 may be of varying size, where the manifold 201 may be smaller than the rigid reservoir 203, or of a size that permits the rigid reservoir 203 to completely encase the manifold 201 with no void space between the rigid reservoir 203 and the manifold 201 when the manifold 201 is positioned within the interstitial space 103 of the rigid reservoir 203. When the manifold 201 is positioned underneath the proximal surface 102 (FIG. 1) of the rigid reservoir 203 the manifold 201 may be of any size that is smaller than the rigid reservoir 203 to allow for the rigid reservoir 203 to cover the top of the manifold 201 and seal the surface and any edges of the manifold 201. In further embodiments, the antimicrobial delivery device may comprise a manifold 201.

In some embodiments, a configurable medicant delivery system may be used to treat a wound such as a burn wound, a dehisced wound, a decubitis ulcer, a diabetic wound, an infected wound, a pressure ulcer, an acute wound, a chronic wound, gangrene, a surgical wound, an ischemic wound, a soft tissue radionecrosis, and combinations thereof.

The manifold 201 may be pre-loaded with a medicant or the like. The medicant may be any anti-microbial agent, anti-fungal agent, or anesthetic agent. Examples of anti-microbial agents that are bacteriocidal may include, but are not limited to, any pencillin, any cephalosporin, any tetracyline, any macrolide, any lincoasmide, any lincoasmine, any sulfonamide, any sulfa drug, any fluoroquinolone, any aminoglycoside, any glycopeptide antibody, any macrolides, any inhibitor of nucleic acid, any topoisomerases, and combinations thereof. Examples of anti-microbial agents that are bacteriostatic may include, but are not limited to, silver, silver compounds, sodium azide, thimerosal, and combinations thereof. In some embodiments, the medicant may be comprised of a poloxamer base and water. Antimicrobial agents, anti-fungal agents or anesthetic agents may then be added. The poloxamer base used may be a polyoxyalkylene based polymer based on ethylene oxide and propylene oxide and comprises a series of closely related block polymers that may generally be classified as polyoxyethylene-polyoxypropylene condensates terminated in primary hydroxyl groups.

In other embodiments, the medicant may be any cytokine or growth factor, such as, but not limited to, Activin A, artemin, chemerin, epidermal growth factor (EGF), fibroblast growth factor 1 (FGF-1), fibroblast growth factor 2 (FGF-2), follistatin, fractalkine, galectin-1, galectin-2, granulocyte macrophage-colony stimulating factor (GM-CSF), interferon gamma-1 (IGF-I), interferon gamma-2 (IGF-II), interleukin 1 alpha (IL-1α), interleukin 1 beta (IL-1β), interleukin 2 (IL-2), interleukin 3 (IL-3), interleukin 4 (IL-4), interleukin 5 (IL-5), interleukin 6 (IL-6), interleukin 7 (IL-7), interleukin 9 (IL-9), interleukin 10 (IL-10), interleukin 11 (IL-11), interleukin 12 (IL-12), interleukin 13 (IL-13), interleukin 15 (IL-15), interleukin 16 (IL-16), interleukin 17 (IL-17), interleukin 19 (IL-19), interleukin 20 (IL-20), interleukin 21 (IL-21), interleukin 22 (IL-22), interleukin 27 (IL-27), interleukin 29 (IL-29), interleukin 31 (IL-31), interleukin 32 alpha (IL-32α), interleukin 33 (IL-33), leptin, macrophage colony-stimulating factor (MCSF), myostatin, nerve growth factor beta (NGF-β), platelet-derived growth factor (PDGF), procalcitonin, tumor necrosis factor alpha (TNF-α), vascular endothelial growth factor (VEGF), and the like and combinations thereof.

In some embodiments, the medicant may be an anti-microbial agent and a growth factor. In other embodiments, the medicant may be a combination of multiple anti-microbial agents and a growth factor. In further embodiments, the medicant may be a combination of multiple anti-microbial agents and multiple growth factors. In yet further embodiments, the medicant may be an anti-microbial agent and multiple growth factors.

Thus, since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes which come within the meaning and range of equivalency of the claims are intended to be embraced therein.

### EXAMPLES

### Example 1

A manifold was composed of a non-compressible polyether polyurethane foam. The foam contained an impermeable layer on the distal surface. The foam was a reticulated foam with efflux pores ranging from 1000 micrometers to 7000 micrometers. The proximal surface of the reservoir was a layer of non-adherent gauze. The manifold was pre-loaded with a poloxamer gel containing the antimicrobial agents Polymyxin B and Nitrofurantoin, and the antifungal agent Nystatin. This antimicrobial delivery reservoir was placed in a wound of a patient, covered with a wound drape, and negative pressure applied through a conduit attached to a first end of a tube which was attached to a source of negative pressure at the second end of the tube.

### Example 2

A rigid reservoir was constructed of plastic. The proximal surface of the reservoir contained a series of efflux pores with a diameter of 2000 micrometers. The size of the efflux pores within the interstitial space varied to control the delivery rate of a medicant. The reservoir contained an injection port through which a medicant was injected into the reservoir. The injection tube contained a valve that with the ability to close. This reservoir was placed into the wound, covered with a wound drape, and negative pressure was created. At any time that the reservoir needs filling with medicant, the valve in the injection tube was opened and medicant was injected from a delivery device, such as a syringe, into the reservoir. Thus the negative pressure was maintained continuously and medicant added as needed.

What is claimed is:

1. An antimicrobial delivery device comprising:
a rigid reservoir having a proximal surface, an interstitial space, and a distal surface; and
a manifold positioned underneath the proximal surface of the rigid reservoir.

2. An antimicrobial delivery device comprising a rigid reservoir having a proximal surface, an interstitial space, and a distal surface, wherein the rigid reservoir comprises a manifold positioned within the interstitial space.

3. The device of claim 2, further comprising a negative pressure conduit positioned to create suction at the distal surface of the rigid reservoir.

4. The device of claim 3, wherein the negative pressure conduit is attached to a first end of a tube and a source of negative pressure is attached to a second end of the tube.

5. The device of claim 2, wherein the manifold is selected from the group consisting of a foam, a sponge, a hydrogel, and a semi-permeable membrane cell.

6. The device of claim 5, wherein the manifold is a foam comprising efflux pores.

7. The device of claim 6, wherein the efflux pores range from about 1 micron to 700 microns.

8. The device of claim 6, wherein the foam has a compressive strength of about 0.05 MPa to about 100 MPa.

9. The device of claim 5, wherein the manifold is a sponge comprising efflux pores.

10. The device of claim 9, wherein the efflux pores range from about micron to 1000 microns.

11. The device of claim 5, wherein the manifold is a hydrogel comprising efflux pores.

12. The device of claim 10, wherein the efflux pores range from about 1 micron to 700 microns.

13. The device of claim 11, wherein the hydrogel is selected from the group consisting of polyvinyl alcohol (PVA) polyhydroxyethyl methacrylate, polyvinyl pyrrolidone, polyacrylamide, polyacrylic acid, hydrolyzed polyacrylonitrile, polyethyleneimine, ethoxylated polyethyleneimine, polyallylamine, polyglycols, and combinations thereof.

14. The device of claim 6, wherein the manifold is a semi-permeable membrane cell comprising efflux pores.

15. The device of claim 14, wherein the efflux pores range from a molecular weight cutoff of about 1,000 kilodaltons to about 1,000,000 kilodaltons.

16. The device of claim 2, wherein the manifold is pre-loaded with a medicant.

17. The device of claim 16, wherein the medicant comprises an anti-microbial agent.

18. The device of claim 16, wherein the medicant comprises a growth factor.

19. The device of claim 16, wherein the medicant comprises an anti-microbial agent and a growth factor.

20. An antimicrobial delivery device comprising:
a rigid reservoir having a proximal surface, an interstitial space, and a distal surface;
a manifold positioned underneath the proximal surface of the rigid reservoir; and
an injection port positioned on the distal surface of the manifold and the rigid reservoir.

21. A method of delivering a medicant to a wound under continuous negative pressure comprising:
sealing a manifold over a suctioned wound surface wherein the manifold is proximal to the wound surface; and
effluxing the medicant while providing continuous negative pressure to the wound from a negative pressure source.

22. A configurable medicant delivery system comprising:
an antimicrobial delivery device, wherein the device comprises a rigid reservoir and a manifold;
a wound, wherein the antimicrobial delivery device is placed over the wound;
a means of generating negative pressure between the antimicrobial delivery device and wound thereby creating suction at the wound; and
delivering the medicant under negative pressure to the wound.

23. The configurable medicant delivery system of claim 22, wherein the manifold is selected from the group consisting of a foam, a sponge, a hydrogel, and a semi-permeable membrane cell.

24. The configurable medicant delivery system of claim 22, wherein the wound is selected from the group consisting of a burn wound, dehisced wound, decubitis ulcer, diabetic wound, infected wound, pressure ulcer, acute wound, chronic wound, gangrene, surgical wound, ischemic wound, soft tissue radionecrosis, and combinations thereof.
